(19) United States
(12) Patent Application Publication
(10) Pub. No.: US 2008/0301885 A1
(43) Pub. Date: Dec. 11, 2008

(54) REVERSIBLY CHANGEABLE HAIR COLOR

(76) Inventors: Doris OBERKOBUSCH, Dusseldorf (DE); Horst HOFFKES, Dusseldorf (DE); Wibke GROSS, Dusseldorf (DE); Melanie MOCH, Dormagen (DE)

Correspondence Address: PAUL & PAUL, 2000 MARKET STREET, Suite 2900, PHILADELPHIA, PA, 19103-3229, US

(21) Appl. No.: 12/144,906

(22) Filed: Jun. 24, 2008

Related U.S. Application Data
(63) Continuation of application No. PCT/EP2006/012379, filed on Dec. 21, 2006.

(30) Foreign Application Priority Data
Dec. 27, 2005 (DE) 10 2005 062 834.6

(51) Int. Cl. A61K 8/40; A61Q 5/00

(52) U.S. Cl. 8/405

(57) ABSTRACT

A coloring system with special CH-acidic compounds in combination with selected aldehydes as reactive carbonyl compound is suitable for obtaining a changeable color. The colors of the fibers colored with this combination are changed and restored again in a pH-controlled manner using appropriate cosmetic compositions.

### CROSS-REFERENCE TO RELATED APPLICATIONS

This application is a continuation under 35 U.S.C. Section 365(c) and 35 U.S.C. Section 120 of International Application No. PCT/EP2006/012379, filed Dec. 21, 2006. This application also claims priority under 35 U.S.C. Section 119 of German Patent Application No. DE 10 2005 062 834.6, filed Dec. 27, 2005.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

(1) Field of the Invention

The invention relates to a composition for the coloring of keratin-containing fibers, in particular, human hair, which comprises special CH-acidic compounds in combination with selected aldehydes as reactive carbonyl compound, to the use of this combination in compositions for obtaining a changeable hair color, and to a method of coloring keratin-containing fibers, in particular, human hair, and reversible change of the color without use of coloring substances.

For the coloring of keratin-containing fibers, use is generally made either of direct dyes or oxidation dyes which are formed by oxidative coupling of one or more developer components with one another or with one or more coupler components. Coupler and developer components are also referred to as oxidation dye precursors.

The developer components used are usually primary aromatic amines with a further free or substituted hydroxy or amino group located in the para or ortho position, diaminopyridine derivatives, heterocyclic hydrazones, 4-aminopyrazolone derivatives, and 2,4,5,6-tetraminopyrimidine and derivatives thereof.

Specific representatives are, for example, p-phenylenediamine, p-tolylenediamine, 2,4,5,6-tetraminopyrimidine, p-aminophenol, N,N-bis(2-hydroxyethyl)-p-phenylenediamine, 2-(2,5-diaminophenyl)ethanol, 2-(2,5-diaminophenoxy)ethanol, 1-phenyl-3-carboxyamido-4-aminopyrazol-5-one, 4-amino-3-methylphenol, 2-aminomethyl-4-aminophenol, 2-hydroxymethyl-4-aminophenol, 2-hydroxy-4,5,6-triaminopyrimidine, 2,4-dihydroxy-5,6-diaminopyrimidine and 2,5,6-triamino-4-hydroxypyrimidine.

The coupler components used are generally m-phenylenediamine derivatives, naphthols, resorcinol and resorcinol derivatives, pyrazolones, m-aminophenols and substituted pyridine derivatives. Suitable coupler substances are, in particular, α-naphthol, 1,5-, 2,7- and 1,7-dihydroxynaphthalene, 5-amino-2-methylphenol, m-aminophenol, resorcinol, resorcinol monomethyl ether, m-phenylenediamine, 2,4-diaminophenoxyethanol, 2-amino-4-(2-hydroxyethylamino)anisol (Lehman n's blue), 1-phenyl-3-methylpyrazol-5-one, 2,4-dichloro-3-aminophenol, 1,3-bis(2,4-diaminophenoxy)propane, 2-chlororesorcinol, 4-chlororesorcinol, 2-chloro-6-methyl-3-aminophenol, 2-methylresorcinol, 5-methylresorcinol, 3-amino-6-methoxy-2-methylaminopyridine and 3,5-diamino-2,6-dimethoxypyridine.

With regard to further customary dye components, reference is made expressly to the “dermatology” series, published by Ch. Culnan, H. Maibach, Verlag Marcel Dekker Inc., New York, Basle, 1986, vol. 7, Ch. Zviak, The Science of Hair Care, chapter 7, pages 248-250 (direct dyes), and chapter 8, pages 264-267 (oxidation dyes), and the “European Inventory of Cosmetics Raw Materials”, 1996, published by the European Commission, available in disk form from the Bundesverband der deutschen Industrie-und Handelsunternehmen für Arzneimittel, Reformwaren und Körperpflegemittel e.V. Mannheim.

Using oxidation dyes, although it is possible to achieve intense colors with good fastness properties, the development of the color generally takes place under the influence of oxidizing agents, such as, for example, H₂O₂, which in some cases can result in fiber damage. The problem continues to be the provision of oxidation hair colors in the red range with adequate fastness properties, in particular, with very good washing and rubbing fastnesses. Furthermore, some oxidation dye precursors and certain mixtures of oxidation dye precursors can sometimes have a sensitizing effect in people with sensitive skin. Direct dyes are applied under relatively gentle conditions, although their disadvantage is that the colors often have only inadequate fastness properties.

(2) Description of Related Art, Including Information Disclosed Under 37 C.F.R. Sections 1.97 and 1.98.

The publication H. Baumann et al., Liebigs Ann. Chem. 1968, 717, 124-136, describes reactions of pyrimidones as methylene bases. A hair colorant comprising 1,2-dihydro-2-oxopyrimidinium derivatives, or the use of the disclosed hemicyanines for the coloring of keratin-containing fibers is not proposed here.

German Patent Application DE-A1-2047431 describes cationic methine dyes for the coloring of anionically modified fibers, such as acidically modified polyesters or acrylonitrile polymers. To synthesize the cationic methine dyes, use is made inter alia of 3,4-dihydro-3-methyl-4-methylenequinazol-2-one and 1,3,6-trimethyl-4-methylenepyrimidin-2-one and obligatorily terephthalaldehyde.

German Patent Application DE-A1-2165913 proposes a method of producing bleaching-out formers using photosensitive dyes. The claimed photosensitive dyes belong to the class of pyrimidone and thio-pyrimidone dyes.

German Patent Application DE-A1-102 41 076 proposes 1,2-dihydro-2-oxopyrimidinium derivatives in combination with reactive carbonyl compounds as agents for coloring keratin fibers.

Often, the consumer deems the color of his hair inappropriate for a certain occasion. In such a situation, the consumer shies away from coloring the hair to fit every occasion. For a colorful masquerade, e.g., for carnival or for a visit to the disco, the consumer desires a hair color which remains unchanged over a certain period and afterward can be returned again to an inconspicuous color or, best of all, to the starting color.

### BRIEF SUMMARY OF THE INVENTION

Surprisingly, it has now been found that on the one hand selected CH-acidic compounds in combination with benzaldehyde derivatives are exceptionally suitable for coloring keratin-containing fibers. They produce colorations with excellent brilliance and color depth and lead to diverse color nuances. Even without the use of oxidizing agents, in particular, colorations with improved washing and light fastness properties over a nuance range from yellow via yellow-brown, orange, brown-orange, brown, red, red-violet to blue-violet, dark blue and black are obtained. Through a rinse, in particular, without the use of coloring components, on the other hand, the coloration achieved can be changed to a second color. This second color can be changed back again to the first color by means of another rinse, in particular, without use of coloring components.

The invention firstly provides a packaging unit (kit) comprising
in a container 1a a composition comprising, in a cosmetic carrier, at least one CH-acidic compound selected from the group which is formed from compounds according to formula I and/or enamine forms thereof, and from compounds of the formula (II), and compounds of the formula (III)

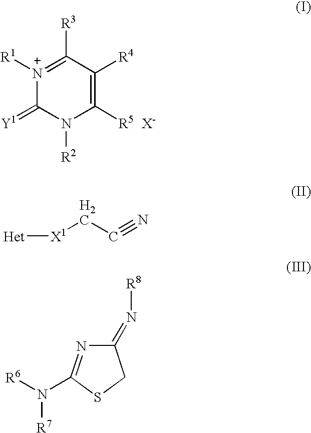

in which
R¹ and R², independently of one another, are a linear or cyclic C₁-C₆-alkyl group, a C₂-C₆-alkenyl group, an optionally substituted aryl group, an optionally substituted heteroaryl group, an aryl-C₁-C₆-alkyl group, a C₁-C₆-hydroxyalkyl group, a C₂-C₆ polyhydroxyalkyl group, a C₁-C₆-alkoxy-C₁-C₆-alkyl group, a group R^{I}R^{II}N—(CH₂)ₚ—, in which R^{I} and R^{II}, independently of one another, are a hydrogen atom, a C₁-C₄-alkyl group, a C₁-C₄-hydroxyalkyl group or an aryl-C₁-C₆-alkyl group, where R^{I} and R^{II}, together with the nitrogen atom, can form a 5-, 6- or 7-membered ring and p is a number 2, 3, 4, 5 or 6,
R³ and R⁵, independently of one another, are a hydrogen atom or a C₁-C₆-alkyl group, where at least one of the radicals R³ or R⁵ is a C₁-C₆ alkyl group,
R⁴ is a hydrogen atom, a C₁-C₆-alkyl group, a C₁-C₆-hydroxyalkyl group, a C₂-C₆ polyhydroxyalkyl group, a C₁-C₆-alkoxy group, a C₁-C₆-hydroxyalkoxy group, a group R^{III}RI^{V}N—(CH₂)_{q}—, in which R^{III}nd R^{IV}, independently of one another, are a hydrogen atom, a C₁-C₆-alkyl group, a C₁-C₆-hydroxyalkyl group or an aryl-C₁-C₆ alkyl group and q is a number 1, 2, 3, 4, 5 or 6, where the radical R⁴, together with one of the radicals R³ or R⁵, can form a 5- or 6-membered aromatic ring which can optionally be substituted by a halogen atom, a C₁-C₆-alkyl group, a C₁-C₆-hydroxyalkyl group, a C₂-C₆-polyhydroxyalkyl group, a C₁-C₆-alkoxy group, a C₁-C₆-hydroxyalkoxy group, a nitro group, a hydroxy group, a group R^{V}R^{VI}N—(CH₂)₈—, in which R^{V} and R^{VI}, independently of one another, are a hydrogen atom, a C₁-C₆-alkyl group, a C₁-C₆-hydroxyalkyl group or an aryl-C₁-C₆ alkyl group and s is a number 0, 1, 2, 3, 4, 5 or 6,
Y¹ is an oxygen atom, a sulfur atom or a group NR^{VII}, in which R^{VII} is a hydrogen atom, an aryl group, a heteroaryl group, a C₁-C₆-alkyl group or a C₁-C₆-arylalkyl group,
X⁻ is a physiologically compatible anion,
Het is an optionally substituted hetero-aromatic,
X¹ is a direct bond or a carbonyl group,
R⁶ and R⁷ form either together with the nitrogen atom a saturated or unsaturated 5- or 6-membered ring or, independently of one another, are a (C₁-C₆)-alkyl group, a (C₂-C₆)-alkenyl group, an aryl group, an aryl-(C₁-C₆)-alkyl group, a (C₂-C₆)-hydroxyalkyl group, a (C₂-C₆)-polyhydroxyalkyl group or a group R^{I}R^{II}N(CH₂)ₘ—, in which R^{I} and R^{II}, independently of one another, are a hydrogen atom, a (C₁-C₆)-alkyl group, a (C₁-C₆)-alkenyl group or an aryl-C₁-C₆-alkyl group, where R^{I} and R^{II}, together with the nitrogen atom, can form a 5-, 6- or 7-membered ring and m is a number 2, 3, 4, 5 or 6, and
R⁸ is a hydrogen atom, a (C₁-C₆)-alkyl group, a (C₂-C₆)-alkenyl group, an aryl group, an aryl-(C₁-C₆)-alkyl group, a (C₂-C₆)-hydroxyalkyl group, a (C₂-C₆)polyhydroxyalkyl group or a group R^{III}R^{IV}N—(CH₂)ₙ—, in which R^{III} and R^{IV}, independently of one another, are a hydrogen atom, a (C₁-C₆)-alkyl group, a (C₁-C₆)-alkenyl group or an aryl-C₁-C₆-alkyl group, where R^{III} and R^{IV}, together with the nitrogen atom, can form a 5-, 6- or 7-membered ring and n is a number 2, 3, 4, 5 or 6,
in a container 1b a composition comprising, in a cosmetic carrier, at least one aldehyde according to formula (IV)

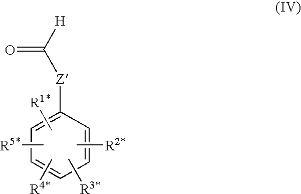

in which
R^{1*}, R^{2*} and R^{3*}, independently of one another, are a hydrogen atom, a halogen atom, a C₁-C₆-alkyl group, a hydroxy group, a C₁-C₆-alkoxy group, a C₁-C₆ dialkylamino group, a di(C₂-C₆-hydroxyalkyl)amino group, a di(C₁-C₆-alkoxy C₁-C₆-alkyl)amino group, a C₁-C₆-hydroxyalkyloxy group, a sulfonyl group, a carboxy group, a sulfonic acid group, a sulfonamido group, a sulfonamide group, carbamoyl group, a C₂-C₆-acyl group or a nitro group
Z′ is a direct bond or a vinylene group,
R^{4*} and R^{5*} are a hydrogen atom or form jointly, together with the remainder of the molecule, a 5- or 6-membered aromatic or aliphatic ring,
in a container 2 a cosmetic composition with an acidic pH and
optionally in a container 3 a cosmetic composition with an alkaline pH.

Suitable cosmetic carriers for all compositions in the kit are generally, for example, creams, emulsions, gels and also surfactant-containing foaming solutions, such as, for example, shampoos, foam aerosols or other preparations which are suitable particularly for use on the hair. However, it is also conceivable to integrate the ingredients into a pulverulent or tablet-like formulation which is dissolved in water prior to use. Preference is given to creams, emulsions and gels.

The carriers are in particular, aqueous or aqueous-alcoholic.

An aqueous carrier comprises at least 50% by weight of water.

### BRIEF DESCRIPTION OF THE SEVERAL VIEWS OF THE DRAWING(S)

Not Applicable

### DETAILED DESCRIPTION OF THE INVENTION

For the purposes of the present invention, aqueous-alcoholic carriers are to be understood as meaning aqueous solutions comprising 3 to 70% by weight of a C₁-C₄-alcohol, in particular, ethanol or isopropanol. The compositions according to the invention can additionally comprise further organic solvents, such as, for example, methoxybutanol, benzyl alcohol, ethyl diglycol or 1,2-propylene glycol. Preference is given here to all water-soluble organic solvents.

Preferably, the compounds according to formula I are selected from one or more compounds of the group of salts with a physiologically compatible counterion X⁻ which is formed from salts of
1,2-dihydro-1,3,4,6-tetramethyl-2-oxopyrimidinium,
1,2-dihydro-1,3-diethyl-4,6-dimethyl-2-oxopyrimidinium,
1,2-dihydro-1,3-dipropyl-4,6-dimethyl-2-oxopyrimidinium,
1,2-dihydro-1,3-di(2-hydroxyethyl)-4,6-dimethyl-2-oxopyrimidinium,
1,2-dihydro-1,3-diphenyl-4,6-dimethyl-2-oxopyrimidinium,
1,2-dihydro-1,3,4-trimethyl-2-oxopyrimidinium,
1,2-dihydro-1,3-diethyl-4-methyl-2-oxopyrimidinium,
1,2-dihydro-1,3-dipropyl-4-methyl-2-oxopyrimidinium,
1,2-dihydro-1,3-di(2-hydroxyethyl)-4-methyl-2-oxopyrimidinium,
1,2-dihydro-1,3-diphenyl-4-methyl-2-oxopyrimidinium,
1-allyl-1,2-dihydro-3,4,6-trimethyl-2-oxopyrimidinium,
1,2-dihydro-1-(2-hydroxyethyl)-3,4,6-trimethyl-2-oxopyrimidinium,
1,2-dihydro-1,3,4,6-tetramethyl-2-thioxopyrimidinium,
1,2-dihydro-1,3-diethyl-4,6-dimethyl-2-thioxopyrimidinium,
1,2-dihydro-1,3-dipropyl-4,6-dimethyl-2-thioxopyrimidinium,
1,2-dihydro-1,3-di(2-hydroxyethyl)-4,6-dimethyl-2-thioxopyrimidinium,
1,2-dihydro-1,3-diphenyl-4,6-dimethyl-2-thioxopyrimidinium,
1,2-dihydro-1,3,4-trimethyl-2-thioxopyrimidinium,
1,2-dihydro-1,3-diethyl-4-methyl-2-thioxopyrimidinium,
1,2-dihydro-1,3-dipropyl-4-methyl-2-thioxopyrimidinium,
1,2-dihydro-1,3-di(2-hydroxyethyl)-4-methyl-2-thioxopyrimidinium,
1,2-dihydro-1,3-diphenyl-4-methyl-2-thioxopyrimidinium,
1,2-dihydro-3,4-dimethyl-2-oxoquinazolinium and
1,2-dihydro-3,4-dimethyl-2-thioxoquinazolinium.

The physiologically compatible anions X⁻ according to formula (I) or of the above-mentioned list must, according to the definition, not only carry one negative charge, but can also have a charge number greater than 1. In the latter case, the anions X⁻ of the salt form to preserve electroneutrality are described by formulating a stoichiometric coefficient of less than 1 in front of the name of the anion. The physiologically compatible anions are preferably selected from halide, 0.5 sulfate, hydrogensulfate, 0.5 carbonate, hydrogencarbonate, ⅓ phosphate, 0.5 hydrogenphosphate, dihydrogenphosphate, carboxylate, such as, for example, lactate, citrate or tartrate. Particularly preferably, X⁻ is chloride, bromide or a carboxylate counterion, in particular, lactate, citrate or tartrate.

If the compounds with the formula (II) according to the invention are compounds that contain nitrogen atoms, in many cases, the known salts can be produced from these in the customary manner as acid addition salts. All statements in this specification and accordingly the claimed protective range therefore refer both to the compounds present in free form and also to their water-soluble, physiologically compatible salts. Examples of such salts are the hydrochlorides, the hydrobromides, the sulfates, the phosphates, the acetates, the propionates, the citrates and the lactates. The hydrochlorides and the sulfates here are particularly preferred. The same is true for compounds containing amino groups according to formula (III).

According to the invention, compounds according to formula (II) that are particularly well suited are those in which the radical Het according to formula II is derived from one of the hetero-aromatics furan, thiophene, pyrrole, isoxazole, isothiazole, imidazole, oxazole, thiazole, pyridine, pyridazine, pyrimidine, pyrazine, 1,2,3-triazine, 1,2,4-triazine, 1,3,5-triazine, benzopyrrole, benzofuran, benzothiophene, benzimidazole, benzoxazole, indazole, benzoisoxazole, benzoisothiazole, indole, quinoline, isoquinoline, cinnoline, phthalazine, quinazoline, quinoxaline, acridine, benzoquinoline, benzoisoquinoline, phenazine, benzocinnoline, benzoquinazoline, benzoquinoxaline, phenoxazine, phenothiazine, nephthyridine, phenanthroline, indolizine, quinolizine, carboline, purine, pteridine and coumarin, where the above-mentioned heteroaromatics can be substituted by at least one group selected from a halogen atom, a nitro group, a thio group, a thio-(C₁-C₆)-alkyl group, a heteroaryl group, an aryl group, a (C₁-C₆)-alkyl group, a (C₁-C₆)-alkoxy group, a hydroxy group, a (C₂-C₆)-hydroxyalkyl group, a (C₂-C₆)-polyhydroxyalkyl group, a (C₁-C₆)-alkoxy-(C₁-C₆)-alkyl group, an aryl-(C₁-C₆)-alkyl group, an amino group, a (C₁-C₆)-monoalkylamino group, a (C₁-C₆)-dialkylamino group, a dialkylaminoalkyl group —(CH₂)ₙ—NR′R″, in which n is an integer from 2 to 6 and R′ and R″, independently of one another, are a linear or branched alkyl group which can optionally together form a ring.

Preferably, the compounds according to formula II are selected from at least one representative of the group consisting of 2-(2-furoyl)acetonitrile, 2-(5-bromo-2-furoyl)acetonitrile, 2-(5-methyl-2-trifluoromethyl-3-furoyl)acetonitrile, 3-(2,5-dimethyl-3-furyl)-3-oxopropanitrile, 2-(2-thenoyl)acetonitrile, 2-(3-thenoyl)acetonitrile, 2-(5-fluoro-2-thenoyl)acetonitrile, 2-(5-chloro-2-thenoyl)acetonitrile, 2-(5-bromo-2-thenoyl)acetonitrile, 2-(5-methyl-2-thenoyl)acetonitrile, 2-(2,5-dimethylpyrrol-3-oyl)acetonitrile, 2-(1,2,5-trimethylpyrrol-3-oyl)acetonitrile, 1H-benzimidazol-2-ylacetonitrile, 1H-benzothiazol-2-ylacetonitrile, 2-(pyrid-2-yl)acetonitrile, 2,6-bis(cyanomethyl)pyridine, 2-(indol-3-oyl)acetonitrile, 2-(2-methylindol-3-oyl)acetonitrile, 8-cyanoacetyl-7-methoxy-4-methylcoumarin, 2-(2-isopropyl-5,6-benzoquinolin-4-oyl)acetonitrile, 2-(2-phenyl-5,6-benzoquinolin-4-oyl)acetonitrile, 2-(quinoxalin-2-yl)acetonitrile, 2-(coumaron-2-yl)acetonitrile, butyl 6,7-dichloro-5-(cyanoacetyl)-2,3-dihydro-1-benzofuran-2-carboxylate, 2-(6-hydroxy-4,7-dimethoxy-1-benzofuran-5-oyl)acetonitrile and 2-(1-phenyl-1,4-dihydrothiochromeno[4,3-c]pyrazol-3-oyl)acetonitrile.

Preferably suitable compounds of the formula (III) are selected from the representatives in which the radicals R⁶ and R⁷ according to formula (III) together with the nitrogen atom form a saturated 5- or 6-membered ring. This ring in turn can optionally contain an oxygen atom and/or optionally two or more nitrogen atoms in the structure. Particularly preferred examples of such rings are piperidinyl, morpholinyl and pyrrolidinyl.

According to the invention, very particularly preferred compounds according to formulae (I), (II) and formula (III) are selected from at least one of the following compounds

| | Structure |
|---|---|
| Salts of 1,2-dihydro-1,3,4,6-tetramethyl-2-oxopyrimidinium | 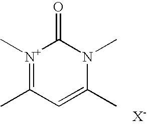 |
| Salts of 1,2-dihydro-1,3,4-trimethyl-2-oxopyrimidinium | 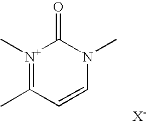 |
| Salts of 1,2-dihydro-1,3,4,6-tetramethyl-2-thioxopyrimidinium | 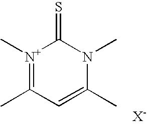 |
| Salts of 1-allyl-1,2-dihydro-3,4,6-trimethyl-2-oxopyrimidinium | 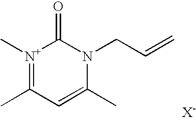 |
| Salts of 1,2-dihydro-1-(2-hydroxyethyl)-3,4,6-trimethyl-2-oxo-pyrimidinium | 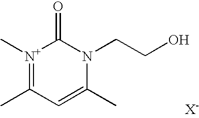 |
| 2-(Cyanomethyl)benzimidazole | 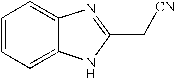 |
| 4,5-Dihydro-4-imino-2-(1-piperidinyl)thiazole | 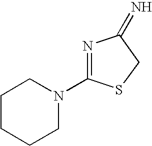 |
| 4,5-Dihydro-4-imino-2-(4-morpholinyl)thiazole | 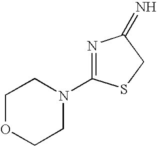 |
| 4,5-Dihydro-4-imino-2-(pyrrolidinyl)thiazole | 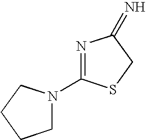 |

where the salts of the above-mentioned compounds contain X⁻ as a physiologically compatible counterion. Very particularly preferred compounds of the formula (I) are salts of 1,2-dihydro-1,3,4,6-tetramethyl-2-oxopyrimidinium (in particular, where X⁻=hydrogensulfate), of 1-allyl-1,2-dihydro-3,4,6-trimethyl-2-oxopyrimidinium (in particular, where X⁻=bromide), and of 1,2-dihydro-1-(2-hydroxyethyl)-3,4,6-trimethyl-2-oxopyrimidinium (in particular, where X⁻=p-toluenesulfonate).

CH-acidic compounds are generally regarded as being those compounds which carry a hydrogen atom bonded to an aliphatic carbon atom where, on account of electron-withdrawing substituents, activation of the corresponding carbon-hydrogen bond is effected. The compounds according to formulae I, II and III according to the invention are CH-acidic compounds.

The compounds of formula (I) are in chemical equilibrium with their corresponding enamine form. With the help of a base it is possible to synthesize the corresponding enamines in a targeted manner from the compounds of said formulae by deprotonation on the carbon atom of the activated methyl groups in position 4 or 6. By way of example, this deprotonation is illustrated below on the radical R³ of the formula I. Compounds according to formula Ia are examples of the enamine forms according to the invention of the compounds according to the invention according to formula (I). Comparable deprotonation on the radical R⁵ of the formula (I) is likewise possible.

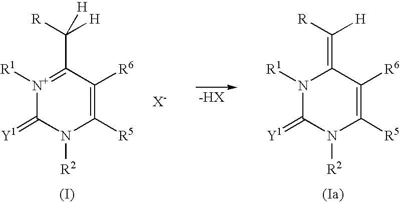

Keratin-containing fibers are to be understood as meaning wool, furs, feathers and in particular, human hair. The colorants according to the invention can, in principle, however also be used for coloring other natural fibers, such as, for example, cotton, jute, sisal, linen or silk, modified natural fibers, such as, for example, regenerated cellulose, nitro-, alkyl- or hydroxyalkyl- or acetylcellulose.

The compounds of the formula (IV) are preferably selected from at least one compound of the group 4-hydroxy-3-methoxybenzaldehyde, 3,5-dimethoxy-4-hydroxybenzaldehyde, 4-hydroxy-1-naphthaldehyde, 4-hydroxy-2-methoxybenzaldehyde, 3,4-dihydroxy-5-methoxybenzaldehyde, 3,4,5-trihydroxybenzaldehyde, 3,5-dibromo-4-hydroxybenzaldehyde, 4-hydroxy-3-nitrobenzaldehyde, 3-bromo-4-hydroxybenzaldehyde, 4-hydroxy-3-methylbenzaldehyde, 3,5-dimethyl-4-hydroxybenzaldehyde, 5-bromo-4-hydroxy-3-methoxybenzaldehyde, 4-diethylamino-2-hydroxybenzaldehyde, 4-dimethylamino-2-methoxybenzaldehyde, 2-methoxybenzaldehyde, 3-methoxybenzaldehyde, 4-methoxybenzaldehyde, 2-ethoxybenzaldehyde, 3-ethoxybenzaldehyde, 4-ethoxybenzaldehyde, 4-hydroxy-2,3-dimethoxybenzaldehyde, 4-hydroxy-2,5-dimethoxybenzaldehyde, 4-hydroxy-2,6-dimethoxybenzaldehyde, 4-hydroxy-2-methylbenzaldehyde, 4-hydroxy-2,3-dimethyl benzaldehyde, 4-hydroxy-2,5-dimethylbenzaldehyde, 4-hydroxy-2,6-dimethylbenzaldehyde, 3,5-diethoxy-4-hydroxybenzaldehyde, 2,6-diethoxy-4-hydroxybenzaldehyde, 3-hydroxy-4-methoxybenzaldehyde, 2-hydroxy-4-methoxybenzaldehyde, 2-ethoxy-4-hydroxybenzaldehyde, 3-ethoxy-4-hydroxybenzaldehyde, 4-ethoxy-2-hydroxybenzaldehyde, 4-ethoxy-3-hydroxybenzaldehyde, 2,3-dimethoxybenzaldehyde, 2,4-dimethoxybenzaldehyde, 2,5-dimethoxybenzaldehyde, 2,6-dimethoxybenzaldehyde, 3,4-dimethoxybenzaldehyde, 3,5-dimethoxybenzaldehyde, 2,3,4-trimethoxybenzaldehyde, 2,3,5-trimethoxybenzaldehyde, 2,3,6-trimethoxybenzaldehyde, 2,4,6-trimethoxybenzaldehyde, 2,4,5-trimethoxybenzaldehyde, 2,5,6-trimethoxybenzaldehyde, 2-hydroxybenzaldehyde, 3-hydroxybenzaldehyde, 4-hydroxybenzaldehyde, 2,3-dihydroxybenzaldehyde, 2,4-dihydroxybenzaldehyde, 2,4-dihydroxy-3-methylbenzaldehyde, 2,4-dihydroxy-5-methylbenzaldehyde, 2,4-dihydroxy-6-methylbenzaldehyde, 2,4-dihydroxy-3-methoxybenzaldehyde, 2,4-dihydroxy-5-methoxybenzaldehyde, 2,4-dihydroxy-6-methoxybenzaldehyde, 2,5-dihydroxybenzaldehyde, 2,6-dihydroxybenzaldehyde, 3,4-dihydroxybenzaldehyde, 3,4-dihydroxy-2-methylbenzaldehyde, 3,4-dihydroxy-5-methylbenzaldehyde, 3,4-dihydroxy-6-methylbenzaldehyde, 3,4-dihydroxy-2-methoxybenzaldehyde, 3,5-dihydroxybenzaldehyde, 2,3,4-trihydroxybenzaldehyde, 2,3,5-trihydroxybenzaldehyde, 2,3,6-trihydroxybenzaldehyde, 2,4,6-trihydroxybenzaldehyde, 2,4,5-trihydroxybenzaldehyde, 2,5,6-trihydroxybenzaldehyde, 4-dimethylaminobenzaldehyde, 4-diethylaminobenzaldehyde, 4-dimethylamino-2-hydroxybenzaldehyde, 4-pyrrolidinobenzaldehyde, 4-morpholinobenzaldehyde, 2-morpholinobenzaldehyde, 4-piperidinobenzaldehyde, 3,5-dichloro-4-hydroxybenzaldehyde, 4-hydroxy-3,5-diiodobenzaldehyde, 3-chloro-4-hydroxybenzaldehyde, 5-chloro-3,4-dihydroxybenzaldehyde, 5-bromo-3,4-dihydroxybenzaldehyde, 3-chloro-4-hydroxy-5-methoxybenzaldehyde, 4-hydroxy-3-iodo-5-methoxybenzaldehyde, 2-methoxy-1-naphthaldehyde, 4-methoxy-1-naphthaldehyde, 2-hydroxy-1-naphthaldehyde, 2,4-dihydroxy-1-naphthaldehyde, 4-hydroxy-3-methoxy-1-naphthaldehyde, 2-hydroxy-4-methoxy-1-naphthaldehyde, 3-hydroxy-4-methoxy-1-naphthaldehyde, 2,4-dimethoxy-1-naphthaldehyde, 3,4-dimethoxy-1-naphthaldehyde, 4-dimethylamino-1-naphthaldehyde, 3-hydroxy-4-nitrobenzaldehyde, 2-hydroxy-3-methoxy-5-nitrobenzaldehyde, 5-nitrovanillin, 2,5-dinitrosalicylaldehyde, 5-bromo-3-nitrosalicylaldehyde, 2-dimethylaminobenzaldehyde, 2-chloro-4-dimethylaminobenzaldehyde, 4-dimethylamino-2-methylbenzaldehyde, 4-diethylaminocinnamaldehyde, 4-dibutylaminobenzaldehyde, 3-allyl-4-hydroxybenzaldehyde, 3-allyl-4-hydroxy-5-methoxybenzaldehyde, 3-allyl-4-hydroxy-5-methylbenzaldehyde, 3-allyl-5-bromo-4-hydroxybenzaldehyde, 3,5-diallyl-4-hydroxybenzaldehyde, 3-allyl-4-hydroxy-5-formylbenzaldehyde (5-allyl-4-hydroxyisophthalaldehyde) and piperonal.

Of suitability as compounds of the formula (III) that are particularly preferably suitable for the purposes of the invention is at least one representative of the group which is formed from 4-hydroxy-3-methoxybenzaldehyde (vanillin), 3-ethoxy-4-hydroxybenzaldehyde (ethylvanillin), 3,5-dimethoxy-4-hydroxybenzaldehyde, 4-hydroxy-1-naphthaldehyde, 4-hydroxy-2-methoxybenzaldehyde, 3,4-dihydroxy-5-methoxybenzaldehyde, 3,4,5-trihydroxybenzaldehyde, 3,5-dibromo-4-hydroxybenzaldehyde, 3-bromo-4-hydroxybenzaldehyde, 4-hydroxy-3-methylbenzaldehyde, 3,5-dimethyl-4-hydroxybenzaldehyde and 5-bromo-4-hydroxy-3-methoxybenzaldehyde (5-bromovanillin).

Very particularly preferably, at least one of the following compounds 4-hydroxy-3-methoxybenzaldehyde (vanillin), 3-ethoxy-4-hydroxybenzaldehyde (ethylvanillin), 3,5-dimethoxy-4-hydroxybenzaldehyde, 4-hydroxy-1-naphthaldehyde and 4-hydroxy-2-methoxybenzaldehyde is suitable as compound according to formula (IV).

These preferred and particularly preferred representatives of the compounds according to formula (IV) are in turn preferably combined with the aforementioned preferred compounds of the formulae (I) and/or (II) and/or (III).

According to the invention, it is preferred that the compositions of container 1 and 1b, or the mixture thereof, do not comprise any further coloring components.

Preferably, the composition with an acidic pH does not comprise any coloring components.

The cosmetic composition with an acidic pH preferably has a pH of from pH 2 to pH 6. In general, it is preferred that this composition additionally comprises at least one buffer system. This serves to stabilize the acidic pH during storage and use. Selection of the buffer system for an acidic pH is not subject to any limitations.

Preferably, the composition with an alkaline pH does not comprise any coloring component.

The cosmetic composition with an alkaline pH preferably has a pH of from pH 8 to pH 11. In general, it is preferred that this composition additionally comprises at least one buffer system. This serves to stabilize the pH during storage and use. Selection of the buffer system for an alkaline pH is not subject to any limitations.

Coloring components for the purposes of the invention are constituents of a composition which, if the composition in question is applied to keratin-containing fibers, brings about a color change, that is visible to the eye, of these keratin-containing fibers. Dyes which color the composition but which do not bring about coloration of the keratin-containing fibers can be present in the compositions of container 2 and of container 3.

The cosmetic compositions of containers 2 and 3 comprise a cosmetic carrier and at least one pH extender.

Suitable pH extenders for establishing an acidic pH are preferably carboxylic acids, in particular, food acids (such as, for example, tartaric acid, citric acid, malic acid or lactic acid), phosphoric acid, sulfuric acid or halohydric acids (such as, for example, hydrochloric acid).

Suitable pH extenders for establishing an alkaline pH are preferably ammonia, alkali metal hydroxides (such as, for example, sodium hydroxide or potassium hydroxide), alkanolamines or basic amino acid, such as, for example, arginine or lysine.

According to the invention, the term alkanolamine is to be understood as meaning organic amine compounds which carry at least one C₂- to C₆-hydroxyalkyl group. The C₂- to C₆-hydroxyalkyl group in turn carries at least one hydroxy group. The alkanolamines according to the invention are preferably primary amines.

2-Hydroxyethyl, 1,3-dihydroxy-2-methylpropan-2-yl, 2-ethyl-1,3-dihydroxypropan-2-yl, 1-hydroxy-2-methylbutan-2-yl, 3-hydroxypropyl and 4-hydroxybutyl, for example, function as C₂- to C₆-hydroxyalkyl group.

Alkanolamines are preferably selected from at least one representative of the group which is formed from 2-aminoethanol (monoethanolamine), monoisopropanolamine, 2-amino-2-methylpropanol, 2-amino-2-methyl-1,3-propanediol, 2-amino-2-ethyl-1,3-propanediol and 2-amino-2-methylbutanol, particularly preferably selected from at least one representative of the group which includes monoethanolamine, 2-amino-2-methylpropanol and 2-amino-2-methyl-1,3-propanediol.

To achieve further and more intense colorations, the compositions according to the invention in container 1a and/or 1b can additionally comprise color boosters. The color boosters are preferably selected from the group consisting of piperidine, piperidine-2-carboxylic acid, piperidine-3-carboxylic acid, piperidine-4-carboxylic acid, pyridine, 2-hydroxypyridine, 3-hydroxypyridine, 4-hydroxypyridine, imidazole, 1-methylimidazole, arginine, histidine, pyrrolidine, proline, pyrrolidone, pyrrolidone-5-carboxylic acid, pyrazole, 1,2,4-triazole, piperazidine, derivatives thereof, and physiologically compatible salts thereof.

The aforementioned color boosters may be used in an amount of in each case 0.03 to 65 mmol, in particular, 1 to 40 mmol, in each case based on 100 g of the total composition.

The colorants according to the invention are mixed prior to use from the composition in container 1a and 1b. They produce intense colors even at physiologically compatible temperatures of less than 45° C. They are therefore particularly suitable for coloring human hair.

The presence of oxidizing agents, e.g., H₂O₂, in at least one of the compositions of container 1a or 1b can be dispensed with. It may, however, in certain circumstances, be desirable to add hydrogen peroxide or other oxidizing agents to the ready-to-use colorant for achieving nuances which are lighter than the keratin-containing fibers to be colored. Oxidizing agents are generally used in an amount of from 0.01 to 6% by weight, based on the application solution. An oxidizing agent preferred for human hair is H₂O₂. Mixtures of two or more oxidizing agents, such as, for example, a combination of hydrogen peroxide and peroxodisulfates of the alkali metal and alkaline earth metals or of iodide ion sources, such as, for example, alkali metal iodides and hydrogen peroxide or the above-mentioned peroxodisulfates, can also be used. The oxidizing agent or the oxidizing agent combination can be used according to the invention in the hair colorant in conjunction with oxidation catalysts. Oxidation catalysts are, for example, metal salts, metal chelate complexes or metal oxides which permit an easy change between two oxidation states of the metal ions. Examples are salts, chelate complexes or oxides of iron, ruthenium, manganese and copper. Further possible oxidation catalysts are enzymes. Suitable enzymes are, for example, peroxidases, which can considerably increase the effect of small amounts of hydrogen peroxide. Furthermore, according to the invention, those enzymes which directly oxidize the oxidation dye precursors with the help of atmospheric oxygen, such as, for example, the laccases, or produce in situ small amounts of hydrogen peroxide and in so doing biocatalytically activate the oxidation of the dye precursors, are suitable. Particularly suitable catalysts for the oxidation of the dye precursors are the 2-electron oxidoreductases in combination with the substrates specific therefor, e.g.,

pyranose oxidase and e.g., D-glucose or galactose,

glucose oxidase and D-glucose,

glycerol oxidase and glycerol,

pyruvate oxidase and pyruvic acid or salts thereof,

alcohol oxidase and alcohol (MeOH, EtOH),

lactate oxidase and lactic acid and salts thereof,

tyrosinase oxidase and tyrosine,

uricase and uric acid or salts thereof,

quinoline oxidase and quinoline,

amino acid oxidase and amino acids.

Furthermore, the compositions according to the invention from containers 1a, 1b, 2 and 3 all comprise active ingredients, additives and auxiliaries known in such preparations. In many cases, the colorants comprise at least one surfactant, where in principle both anionic and also zwitterionic, ampholytic, nonionic and cationic surfactants are suitable. In many cases, however, it has proven advantageous to select the surfactants from anionic, zwitterionic or nonionic surfactants.

Suitable anionic surfactants in preparations according to the invention are all anionic surface-active substances suitable for use on the human body. These are characterized by a water-solubilizing, anionic group, such as, for example, a carboxylate, sulfate, sulfonate or phosphate group and a lipophilic alkyl group having about 10 to 22 carbon atoms. Additionally, glycol or polyglycol ether groups, ester, ether and amide groups and also hydroxyl groups may be present in the molecule. Examples of suitable anionic surfactants are, in each case in the form of the sodium, potassium and ammonium and also the mono-, di- and trialkanolammonium salts having 2 or 3 carbon atoms in the alkanol group,
linear fatty acids having 10 to 22 carbon atoms (soaps),
ether carboxylic acids of the formula R-O—(CH₂—CH₂O)ₓ—CH₂—COOH, in which R is a linearalkyl group having 10 to 22 carbon atoms and x=0 or 1 to 16,
acyl sarcosides having 10 to 18 carbon atoms in the acyl group,
acyl taurides having 10 to 18 carbon atoms in the acyl group,
acyl isethionates having 10 to 18 carbon atoms in the acyl group,
sulfosuccinic acid mono- and dialkyl esters having 8 to 18 carbon atoms in the alkyl group and sulfosuccinic acid monoalkyl polyoxyethyl esters having 8 to 18 carbon atoms in the alkyl group and 1 to 6 oxyethyl groups,
linear alkanesulfonates having 12 to 18 carbon atoms,
linear alpha-olefinsulfonates having 12 to 18 carbon atoms,
alpha-sulfo fatty acid methyl esters of fatty acids having 12 to 18 carbon atoms,
alkyl sulfates and alkyl polyglycol ether sulfates of the formula R-O(CH₂—CH₂O)ₓ—SO₃H, in which R is a preferably linear alkyl group having 10 to 18 carbon atoms and x=0 or 1 to 12,
mixtures of surface-active hydroxysulfonates as in DE-A-37 25 030,
sulfated hydroxyalkyl polyethylene and/or hydroxyalkylene propylene glycol ethers as in DE-A-37 23 354,
sulfonates of unsaturated fatty acids having 12 to 24 carbon atoms and 1 to 6 double bonds as in DE-A-39 26 344,
esters of tartaric acid and citric acid with alcohols, which constitute addition products of from about 2 to 15 molecules of ethylene oxide and/or propylene oxide onto fatty alcohols having 8 to 22 carbon atoms.

Preferred anionic surfactants are alkyl sulfates, alkylpolyglycol ether sulfates and ether carboxylic acids having 10 to 18 carbon atoms in the alkyl group and up to 12 glycol ether groups in the molecule, and in particular, salts of saturated and in particular, unsaturated C₈-C₂₂-carboxylic acids, such as oleic acid, stearic acid, isostearic acid and palmitic acid.

Zwitterionic surfactants is the term used to refer to those surface-active compounds which carry at least one quaternary ammonium group and at least one —COO⁽⁻⁾ or —SO₃⁽⁻⁾ group in the molecule. Particularly suitable zwitterionic surfactants are the betaines, such as the N-alkyl-N,N-dimethylammonium glycinates, for example cocoalkyldimethylammonium glycinate, N-acylaminopropyl-N,N-dimethylammonium glycinates, for example cocoacylaminopropyldimethylammonium glycinate, and 2-alkyl-3-carboxymethyl-3-hydroxyethylimidazolines having in each case 8 to 18 carbon atoms in the alkyl or acyl group, and also cocoacylaminoethyl hydroxyethylcarboxymethyl glycinate. A preferred zwitterionic surfactant is the fatty acid amide derivative known under the CTFA name Cocamidopropyl Betaine.

Ampholytic surfactants are understood as meaning those surface-active compounds which, apart from a C₈₋₁₈-alkyl or -acyl group in the molecule, contain at least one free amino group and at least one —COOH or —SO₃H group in the molecule and are capable of forming internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids having in each case about 8 to 18 carbon atoms in the alkyl group. Particularly preferred ampholytic surfactants are N-cocoalkylaminopropionate, cocoacylaminoethylaminopropionate and C₁₂₋₁₈-acylsarcosine.

Nonionic surfactants contain, as hydrophilic group, e.g., a polyol group, a polyalkylene glycol ether group or a combination of polyol and polyglycol ether group. Such compounds are, for example,
addition products of from 2 to 30 mol of ethylene oxide and/or 0 to 5 mol of propylene oxide onto linear fatty alcohols having 8 to 22 carbon atoms, onto fatty acids having 12 to 22 carbon atoms and onto alkylphenols having 8 to 15 carbon atoms in the alkyl group,
C₁₂₋₂₂-fatty acid mono- and diesters of addition products of from 1 to 30 mol of ethylene oxide onto glycerol,
C₈₋₂₂-alkyl mono- and oligoglycosides and ethoxylated analogs thereof,
addition products of from 5 to 60 mol of ethylene oxide onto castor oil and hydrogenated castor oil,
addition products of ethylene oxide onto sorbitan fatty acid esters,
addition products of ethylene oxide onto fatty acid alkanolamides.

Examples of the cationic surfactants that can be used in the compositions according to the invention are, in particular, quaternary ammonium compounds. Preference is given to ammonium halides, such as alkyltrimethylammonium chlorides, dialkyldimethylammonium chlorides and trialkylmethylammonium chlorides, e.g., cetyltrimethylammonium chloride, stearyltrimethylammonium chloride, distearyldimethylammonium chloride, lauryldimethylammonium chloride, lauryldimethylbenzylammonium chloride and tricetylmethylammonium chloride. Further cationic surfactants that can be used according to the invention are the quaternized protein hydrolyzates.

Likewise suitable according to the invention are cationic silicone oils, such as, for example, the commercially available products Q2-7224 (manufacturer: Dow Corning; a stabilized trimethylsilylamodimethicone), Dow Corning 929 emulsion (comprising a hydroxylamino-modified silicone, which is also referred to as amodimethicone), SM-2059 (manufacturer: General Electric), SLM-55067 (manufacturer: Wacker), and Abil®-Quat 3270 and 3272 (manufacturer: Th. Goldschmidt; diquaternary polydimethylsiloxanes, quaternium-80).

Alkylamidoamines, in particular, fatty acid amidoamines, such as the stearylamidopropyldimethylamine available under the name Tego Amid®S 18, are characterized specifically by their good biodegradability besides a good conditioning effect.

Likewise of very good biodegradability are quaternary ester compounds, “ester quats,” such as the methylhydroxyalkyldialkoyloxyalkylammonium methosulfates sold under the trade name Stepantex®.

One example of a quaternary sugar derivative that can be used as cationic surfactant is the commercial product Glucquat®100, according to CTFA nomenclature a “Lauryl Methyl Gluceth-10 Hydroxypropyl Dimonium Chloride.”

The compounds with alkyl groups used as surfactants may in each case be single substances. However, it is generally preferred to start from native vegetable or animal raw materials in the production of these substances, meaning that substance mixtures with different alkyl chain lengths that depend on the particular raw material are obtained.

As regards the surfactants which constitute addition products of ethylene oxide and/or propylene oxide onto fatty alcohols or derivatives of these addition products, it is possible to use either products with a “normal” homolog distribution, or those with a narrowed homolog distribution. In this connection, “normal” homolog distribution is understood as meaning mixtures of homologs which are obtained in the reaction of fatty alcohol and alkylene oxide using alkali metals, alkali metal hydroxides or alkali metal alkoxides as catalysts. Narrowed homolog distributions, on the other hand, are obtained if, for example, hydrotalcites, alkaline earth metal salts of ether carboxylic acids, alkaline earth metal oxides, hydroxides or alkoxides are used as catalysts. The use of products with a narrowed homolog distribution may be preferred.

Further active ingredients, auxiliaries and additives are, for example,
nonionic polymers, such as, for example, vinylpyrrolidone/vinyl acrylate copolymers, polyvinylpyrrolidone and vinylpyrrolidone/vinyl acetate copolymers and polysiloxanes,
cationic polymers, such as quaternized cellulose ethers, polysiloxanes with quaternary groups, dimethyldiallylammonium chloride polymers, acrylamide-dimethyldiallylammonium chloride copolymers, dimethylaminoethyl methacrylate-vinylpyrrolidone copolymers quaternized with diethyl sulfate, vinylpyrrolidone-imidazolinium methochloride copolymers and quaternized polyvinyl alcohol,
zwitterionic and amphoteric polymers, such as, for example, acrylamidopropyltrimethylammonium chloride/acrylate copolymers and octylacrylamide/methyl methacrylate/tert-butylaminoethyl methacrylate/2-hydroxypropyl methacrylate copolymers,
anionic polymers, such as, for example, polyacrylic acids, crosslinked polyacrylic acids, vinyl acetate/crotonic acid copolymers, vinylpyrrolidone/vinyl acrylate copolymers, vinyl acetate/butyl maleate/isobornyl acrylate copolymers, methyl vinyl ether/maleic anhydride copolymers and acrylic acid/ethyl acrylate/N-tert-butylacrylamide terpolymers,
thickeners, such as agar agar, guar gum, alginates, xanthan gum, gum arabic, karaya gum, carob seed flour, linseed gums, dextrans, cellulose derivatives, e.g., methylcellulose, hydroxyalkylcellulose and carboxymethylcellulose, starch fractions and derivatives, such as amylose, amylopectin and dextrins, clays, such as, for example, bentonite, or completely synthetic hydrocolloids, such as, for example, polyvinyl alcohol,
structurants, such as glucose and maleic acid,
hair-conditioning compounds, such as phospholipids, for example soya lecithin, egg lecithin and cephalins, and also silicone oils,
protein hydrolyzates, in particular, elastin, collagen, keratin, milk protein, soya protein and wheat protein hydrolysates, condensation products thereof with fatty acids, and quaternized protein hydrolysates,
perfume oils, dimethyl isosorbide and cyclodextrins,
solubility promoters such as ethanol, isopropanol, ethylene glycol, propylene glycol, glycerol and diethylene glycol,
antidandruff active ingredients such as piroctone olamine and zinc omadine,
further substances for adjusting the pH,
active ingredients, such as panthenol, pantothenic acid, allantoin, pyrrolidone carboxylic acids and salts thereof, plant extracts and vitamins,
cholesterol,
photoprotective agents,
consistency regulators, such as sugar esters, polyol esters or polyol alkyl ethers,
fats and waxes, such as spermaceti, beeswax, montan wax, paraffins, fatty alcohols and fatty acid esters,
fatty acid alkanolamides,
complexing agents, such as EDTA, NTA and phosphonic acids,
swelling and penetration substances, such as glycerol, propylene glycol monoethyl ethers, carbonates, hydrogencarbonates, guanidines, ureas, and primary, secondary and tertiary phosphates, imidazoles, tannins, pyrrole,
opacifiers, such as latex,
pearlizing agents, such as ethylene glycol mono- and distearate,
propellants, such as propane/butane mixtures, N₂O, dimethyl ether, CO₂ and air, and
antioxidants.

The constituents of the cosmetic carrier are used in amounts customary for this purpose for producing the compositions of the packaging unit according to the invention; e.g., emulsifiers are used in concentrations of from 0.5 to 30% by weight and thickeners are used in concentrations of from 0.1 to 25% by weight of the total colorant.

For the coloring result, it may be advantageous to add ammonium or metal salts to at least one composition of container C1a or C1b. Suitable metal salts are, for example, formates, carbonates, halides, sulfates, butyrates, valerates, caproates, acetates, lactates, glycolates, tartrates, citrates, gluconates, propionates, phosphates and phosphonates of alkali metals, such as potassium, sodium or lithium, alkaline earth metals, such as magnesium, calcium, strontium or barium, or of aluminum, manganese, iron, cobalt, copper or zinc, where sodium acetate, lithium bromide, calcium bromide, calcium gluconate, zinc chloride, zinc sulfate, magnesium chloride, magnesium sulfate, ammonium carbonate, ammonium chloride and ammonium acetate are preferred. These salts are preferably present in an amount of from 0.03 to 65 mmol, in particular, from 1 to 40 mmol, based on 100 g of the application mixture.

The pH of the application mixture of the compositions of container C1a and C1b is usually between 2 and 11, preferably between 8 and 10.

The invention secondly provides a method for the reversible recoloring of keratin-containing fibers, in particular, human hair, which have been colored previously with a colorant, comprising, in a cosmetic carrier, a combination of component

(A) at least one compound of the formula (I) and/or (II) and/or (III)

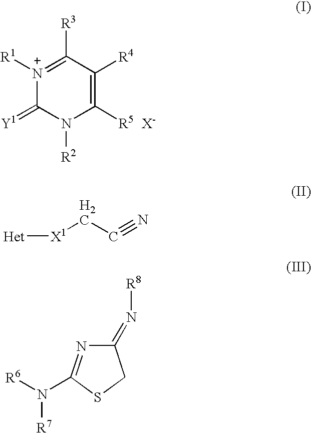

in which R¹, R², R³, R⁴, R⁵, R⁶, R⁷, R⁸, Het, X¹ and Y¹ are as defined in the first subject matter of the invention

with component (B) at least one compound according to formula (IV)

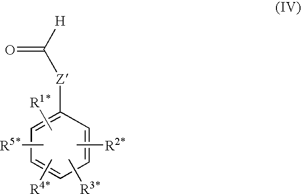

in which R^{1*}, R^{2*}, R^{3*}, R^{4*}, R^{5*} and Z′ are as defined in the first subject matter of the invention,
(a) the colored keratin-containing fibers are recolored using a cosmetic composition with an acidic pH and
(b) optionally after a period of up to 4 weeks, are recolored again using a cosmetic composition with an alkaline pH.

It is preferred if the colorant of the method according to the invention has a pH of from pH 6 to pH 11, particularly preferably a pH of from 8 to 10.

For use in the colorant, the preferred representatives of the compounds of the formulae (I), (II), (III) and (IV) according to the first subject matter of the invention are particularly suitable. All of the preferred embodiments of the compositions of containers 1a and 1b, or the mixture thereof, which are mentioned in the first subject matter of the invention, apply for the colorant of the method according to the invention.

The cosmetic composition with an acidic pH according to step (a) of the method according to the invention has a preferred pH of from pH 2 to pH 6.

For the cosmetic composition from step (a), the preferred and optional parameters of the composition in container 2 of the first subject matter of the invention apply.

The cosmetic composition with an alkaline pH according to step (b) of the method according to the invention has a preferred pH of from pH 8 to pH 11.

For the cosmetic composition from step (b), the preferred and optional parameters of the composition in container 3 of the first subject matter of the invention apply.

After carrying out step (b), the color or shade is preferably achieved which was present before carrying out step (a).

Steps (a) and (b) can be passed through several times.

The fibers colored previously with said colorant can have been colored at any time before carrying out step (a). It is important that a visible color as the result of said colorant exists, so that an effective color change according to step (a) can take place.

According to step (a) or step (b), the corresponding cosmetic composition is applied to the wet or dry keratin-containing fibers. The compositions of steps (a) and (b) can, following application to the colored keratin-containing fibers, either be left on the fibers (leave on) or be rinsed out of the fibers. It is preferred according to the invention to carry out the respective steps (a) or (b) of the method according to the invention without subsequent rinse operation in order to leave the corresponding compositions on the hair.

The invention thirdly provides the use of a cosmetic composition with an acidic pH for changing the color of keratin-containing fibers, in particular, human hair, which have been colored using a colorant comprising, in a cosmetic carrier, a combination of component (A) at least one compound of the formula (I) and/or (II) and/or (III)

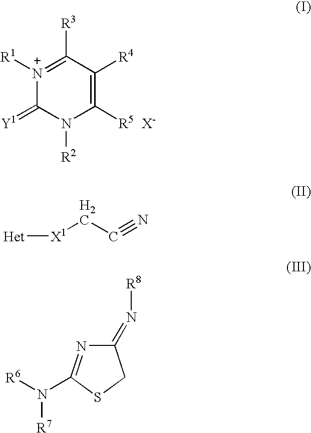

in which R¹, R², R³, R⁴, R⁵, R⁶, R⁷, R⁸, Het, X¹ and Y¹ are as defined in the first subject matter of the invention

with component (B) at least one compound according to formula (IV)

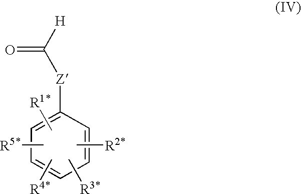

in which R^{1*}, R^{2*}, R^{3*}, R^{4*}, R^{5*} and Z′ are defined as described in the first subject matter of the invention.

The invention fourthly provides the use of a cosmetic composition with an alkaline pH for restoring a color of keratin-containing fibers, in particular, human hair, which have firstly been colored using a colorant comprising, in a cosmetic carrier, a combination of component (A) at least one compound of the formula (I) and/or (II) and/or (III)

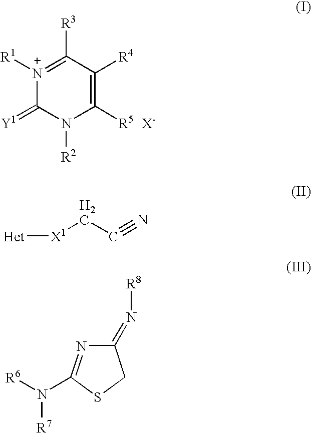

in which R¹, R², R³, R⁴, R⁵, R⁶, R⁷, R⁸, Het, X¹ and Y¹ are defined as in the first subject matter of the invention

with component (B) at least one compound according to formula (IV)

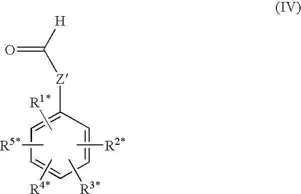

in which R^{1*}, R^{2*}, R^{3*}, R^{4*}, R^{5*} and Z′ are defined as described in the first subject matter of the invention,

and have then been subjected to a color change using a cosmetic composition with an acidic pH.

All preferred and optional parameters of the first and second subject matter of the invention apply mutatis mutandis also for subject matters three and four of the invention.

### EXAMPLES

1.0 Preparation of the Colorants.

| Aqueous gel formulation for component A | Gel 1: | |
|---|---|---|
| CH-acidic compound (component A) | 10 | mmol |
| Natrosol ® HR 250 | 2 | g |
| Water, demineralized | ad 100 | g |

The CH-acidic compound (component A) is firstly dissolved with stirring in a small amount of water, then topped up to 98 g with water. With stirring, the Natrosol (INCI name: Hydroxyethylcellulose; Hercules) is added and one waits until the desired thickening results.

| Aqueous gel formulation for component B | Gel 2: | |
|---|---|---|
| Carbonyl compound (component B) | 10 | mmol |
| Natrosol ® HR 250 | 2 | g |
| NaOH (50% strength aqueous solution) | possibly a few drops | |
| Water, demineralized | ad 100 | g |

The carbonyl compound (component B) is dissolved or suspended in a small amount of water. To increase the solubility, if required, the mixture is alkalized with a few drops of 50% strength sodium hydroxide solution. The mixture is then topped up to 98 g with water and stirred until dissolution of the carbonyl compound is complete (sometimes with gentle heating to about 40° C.). Then, with stirring, the Natrosol is added and the swelling process awaited.

2.0 Colorations.

Aqueous gel formulations from point 1.0 (gel 1 and gel 2) with components A and B as in table 1 were prepared. The gels were mixed in the weight ratio 1:1, then the pH was adjusted to a value of 9 using ammonia or tartaric acid. The dye precursors were used in the combinations listed in Table 1.

The resulting ready-to-use hair colorant was applied to a hair tress of 90% gray, non-pretreated human hair (liquor weight ratio: gel mixture to hair=2 to 1) and evenly distributed using an applicette. After a contact time of 30 minutes at 32° C., the tress was rinsed with lukewarm water and then dried in a warm stream of air (30° C. to 40° C.). The colorations are shown in Table 1.

3.0 Acidic Rinse.

Each colored hair tress from point 2.0 was then rinsed with in each case an aqueous solution adjusted to a pH of pH 3 and then dried in a warm stream of air. The color of the hair was evaluated again and can be found in Table 1.

4.0 Alkaline Rinse.

Each recolored hair tress from point 3.0 was then rinsed with in each case an aqueous solution adjusted to a pH of pH 9, and then dried in a warm stream of air. The color of the hair was evaluated again and can be found in Table 1.

Compounds of component A (Table 1).
A1 1,2-dihydro-1,3,4,6-tetramethyl-2-oxopyrimidinium hydrogensulfate

Compounds of component B (Table 1).
B1 4-hydroxy-3-methoxybenzaldehyde (vanillin)
B2 3-ethoxy-4-hydroxybenzaldehyde (ethylvanillin)
B3 3,5-dimethoxy-4-hydroxybenzaldehyde
B4 4-hydroxy-2-methoxybenzaldehyde
B5 4-hydroxy-1-naphthaldehyde
B6 3,4-dihydroxybenzaldehyde
B7 2,4-dihydroxybenzaldehyde

TABLE 1

| | | | | After |
|---|---|---|---|---|
| Component A | Component B | Initial | After acidic | alkaline |
| (gel 1) | (gel 2) | color | rinse | rinse |
| A1 | B1 | violet | olive brown | Violet |
| A1 | B2 | violet | olive brown | violet |
| A1 | B3 | blue-violet | mid-brown | blue-violet |
| A1 | B4 | red | yellow-orange | red |
| A1 | B5 | dark blue | beige | dark blue |
| A1 | B6 | violet | brown-yellow | violet |
| A1 | B7 | red | yellow | red |

3.0 UV/vis-spectroscopic measurement of the coloring solutions at various pHs

0.0015 mol of component A1 (1,2-dihydro-1,3,4,6-tetramethyl-2-oxopyrimidinium hydrogensulfate) were dissolved in 30 ml of distilled water. Then, 0.0015 ml of an aromatic aldehyde (component B) described under point 2.0 were dissolved or suspended in 30 ml of distilled water. Both solutions were combined, and a pH of 9 was established using ammonia solution. The solution was then stored for 30 minutes at about 30° C.

The solution was brought to a pH of 3 using dilute hydrochloric acid and, following suitable dilution, measured by means of UV/vis spectroscopy. After establishing a pH of 9 with dilute sodium hydroxide solution, the solution was suitably diluted a second time and measured by means of UV/vis spectroscopy. The UV/vis spectra were recorded in the wavelength range from 300 to 700 nm. The λₘₐₓ values listed in table 2 reflect the color shifts that arise as a function of the pH.

TABLE 2

| | | λₘₐₓ (pH = 3) | λₘₐₓ (pH = 9) |
|---|---|---|---|
| Component A | Component B | [300-700 nm] | [300-700 nm] |
| A1 | B1 | 311; 427 | 317; 350; 542 |
| A1 | B3 | 306; 434 | 310; 363; 571 |
| A1 | B4 | 313; 440 | 319; 528 |
| A1 | B6 | 312; 428 | 316; 346; 536 |
| A1 | B7 | 310; 442 | 318; 538 |

1. A kit comprising: (a) a first in container 1a comprising a composition comprising a cosmetic carrier, at least one CH-acidic compound selected from the group consisting of a compound of the formula I and/or enamine forms thereof, a compound of the formula (II), and a compound of the formula (III) 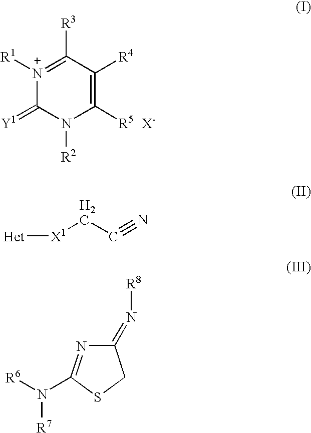
wherein
each of R¹ and R² is independently a linear or cyclic C₁-C₆-alkyl group, a C₂-C₆-alkenyl group, an optionally substituted aryl group, an optionally substituted heteroaryl group, an aryl-C₁-C₆-alkyl group, a C₁-C₆-hydroxyalkyl group, a C₂-C₆-polyhydroxyalkyl group, a C₁-C₆-alkoxy-C₁-C₆-alkyl group, a group R^{I}R^{II}N—(CH₂)ₚ—, in which each if R^{I} and R^{II} is independently a hydrogen atom, a C₁-C₄-alkyl group, a C₁-C₄-hydroxyalkyl group or an aryl-C₁-C₆-alkyl group, where R^{I} and R^{II}, together with the nitrogen atom, can form a 5-, 6- or 7-membered ring and p is a number 2, 3, 4, 5 or 6,
each of R³ and R⁵ is independently a hydrogen atom or a C₁-C₆-alkyl group, wherein at least one of the radicals R³ or R⁵ is a C₁-C₆-alkyl group,
R⁴ is a hydrogen atom, a C₁-C₆-alkyl group, a C₁-C₆-hydroxyalkyl group, a C₂-C₆-polyhydroxyalkyl group, a C₁-C₆-alkoxy group, a C₁-C₆-hydroxyalkoxy group, a group R^{III}R^{IV}N—(CH₂)_{q}—, wherein each of R^{III} and R^{IV} is independently a hydrogen atom, a C₁-C₆-alkyl group, a C₁-C₆-hydroxyalkyl group or an aryl-C₁-C₆-alkyl group and q is a number 1, 2, 3, 4, 5 or 6, where the radical R⁴, together with one of the radicals R³ or R⁵, can form a 5- or 6-membered aromatic ring which can optionally be substituted by a halogen atom, a C₁-C₆-alkyl group, a C₁-C₆-hydroxyalkyl group, a C₂-C₆-polyhydroxyalkyl group, a C₁-C₆-alkoxy group, a C₁-C₆-hydroxyalkoxy group, a nitro group, a hydroxy group, a group R^{V}R^{VI}N—(CH₂)₈—, wherein each of R^{V} and R^{VI} is independently a hydrogen atom, a C₁-C₆-alkyl group, a C₁-C₆-hydroxyalkyl group or an aryl-C₁-C₆-alkyl group and s is a number 0, 1, 2, 3, 4, 5 or 6,
Y¹ is an oxygen atom, a sulfur atom or a group NR^{VII}, wherein R^{VII} is a hydrogen atom, an aryl group, a heteroaryl group, a C₁-C₆-alkyl group or a C₁-C₆-arylalkyl group,
X⁻ is a physiologically compatible anion,
Het is an optionally substituted heteroaromatic,
X¹ is a direct bond or a carbonyl group,
each of R⁵ and R⁷ together with the nitrogen atom to which they are bonded form a saturated or unsaturated 5- or 6-membered ring or, independently of one another, are a (C₁-C₆)-alkyl group, a (C₂-C₆)-alkenyl group, an aryl group, an aryl-(C₁-C₆)-alkyl group, a (C₂-C₆)-hydroxyalkyl group, a (C₂-C₆)-polyhydroxyalkyl group or a group R^{I}R^{II}N—(CH₂)ₘ—, wherein each of R^{I} and R^{II} is independently a hydrogen atom, a (C₁-C₆)-alkyl group, a (C₁-C₆)-alkenyl group or an aryl-C₁-C₆-alkyl group, wherein R^{I} and R^{II}, together with the nitrogen atom to which they are bonded form a 5-, 6- or 7-membered ring and m is a number 2, 3, 4, 5 or 6, and
R⁸ is a hydrogen atom, a (C₁-C₆)-alkyl group, a (C₂-C₆)-alkenyl group, an aryl group, an aryl-(C₁-C₆)-alkyl group, a (C₂-C₆)-hydroxyalkyl group, a (C₂-C₆)-polyhydroxyalkyl group or a group R^{III}R^{IV}N—(CH₂)ₙ—, in which R^{III} and R^{IV}, independently of one another, are a hydrogen atom, a (C₁-C₆)-alkyl group, a (C₁-C₆)-alkenyl group or an aryl-C₁-C₆-alkyl group, where R^{III} and R^{IV}, together with the nitrogen atom, can form a 5-, 6- or 7-membered ring and n is a number 2, 3, 4, 5 or 6;
(b) a second container 1b comprising a cosmetic carrier and at least one aldehyde of the formula (IV) 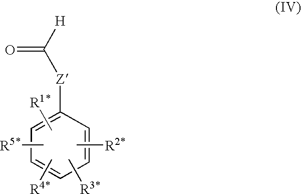
wherein
each of R^{1*}, R^{2*} and R^{3*} is independently a hydrogen atom, a halogen atom, a C₁-C₆-alkyl group, a hydroxy group, a C₁-C₆-alkoxy group, a C₁-C₆-dialkylamino group, a di(C₂-C₆-hydroxyalkyl)amino group, a di(C₁-C₆-alkoxy-C₁-C₆-alkyl)amino group, a C₁-C₆-hydroxyalkyloxy group, a sulfonyl group, a carboxy group, a sulfonic acid group, a sulfonamido group, a sulfonamide group, carbamoyl group, a C₂-C₆-acyl group or a nitro group,
Z′ is a direct bond or a vinylene group,
each of R^{4*} and R^{5*} is a hydrogen atom or together with the remainder of the molecule jointly form a 5- or 6-membered aromatic or aliphatic ring,
wherein the composition in the second container has an acidic pH and;
(c) optionally a third container 3 comprising a cosmetic composition having an alkaline pH.

2. The kit of claim 1 wherein the compound of formula I is selected from the group consisting of:
1,2-dihydro-1,3,4,6-tetramethyl-2-oxopyrimidinium,
1,2-dihydro-1,3-diethyl-4,6-dimethyl-2-oxopyrimidinium,
1,2-dihydro-1,3-dipropyl-4,6-dimethyl-2-oxopyrimidinium,
1,2-dihydro-1,3-di(2-hydroxyethyl)-4,6-dimethyl-2-oxopyrimidinium,
1,2-dihydro-1,3-diphenyl-4,6-dimethyl-2-oxopyrimidinium,
1,2-dihydro-1,3,4-trimethyl-2-oxopyrimidinium,
1,2-dihydro-1,3-diethyl-4-methyl-2-oxopyrimidinium,
1,2-dihydro-1,3-dipropyl-4-methyl-2-oxopyrimidinium,
1,2-dihydro-1,3-di(2-hydroxyethyl)-4-methyl-2-oxopyrimidinium,
1,2-dihydro-1,3-diphenyl-4-methyl-2-oxopyrimidinium,
1-allyl-1,2-dihydro-3,4,6-trimethyl-2-oxopyrimidinium,
1,2-dihydro-1-(2-hydroxyethyl)-3,4,6-trimethyl-2-oxopyrimidinium,
1,2-dihydro-1,3,4,6-tetramethyl-2-thioxopyrimidinium,
1,2-dihydro-1,3-diethyl-4,6-dimethyl-2-thioxopyrimidinium,
1,2-dihydro-1,3-dipropyl-4,6-dimethyl-2-thioxopyrimidinium,
1,2-dihydro-1,3-di(2-hydroxyethyl)-4,6-dimethyl-2-thioxopyrimidinium,
1,2-dihydro-1,3-diphenyl-4,6-dimethyl-2-thioxopyrimidinium,
1,2-dihydro-1,3,4-trimethyl-2-thioxopyrimidinium,
1,2-dihydro-1,3-diethyl-4-methyl-2-thioxopyrimidinium,
1,2-dihydro-1,3-dipropyl-4-methyl-2-thioxopyrimidinium,
1,2-dihydro-1,3-di(2-hydroxyethyl)-4-methyl-2-thioxopyrimidinium,
1,2-dihydro-1,3-diphenyl-4-methyl-2-thioxopyrimidinium,
1,2-dihydro-3,4-dimethyl-2-oxoquinazolinium and
1,2-dihydro-3,4-dimethyl-2-thioxoquinazolinium.
and a salt thereof having a physiologically compatible counterion X⁻.

3. The kit of claim 1 wherein the radical Het of the compound of the formula (II) is derived from a heteroaromatic compound selected from the group consisting of: furan, thiophene, pyrrole, isoxazole, isothiazole, imidazole, oxazole, thiazole, pyridine, pyridazine, pyrimidine, pyrazine, 1,2,3-triazine, 1,2,4-triazine, 1,3,5-triazine, benzopyrrole, benzofuran, benzothiophene, benzimidazole, benzoxazole, indazole, benzoisoxazole, benzoisothiazole, indole, quinoline, isoquinoline, cinnoline, phthalazine, quinazoline, quinoxaline, acridine, benzoquinoline, benzoisoquinoline, phenazine, benzocinnoline, benzoquinazoline, benzoquinoxaline, phenoxazine, phenothiazine, nephthyridine, phenanthroline, indolizine, quinolizine, carboline, purine, pteridine and coumarin; and the heteroaromatics substituted by at least one group selected from the group consisting of a halogen atom, a nitro group, a thio group, a thio-(C₁-C₆)-alkyl group, a heteroaryl group, an aryl group, a (C₁-C₆)-alkyl group, a (C₁-C₆)-alkoxy group, a hydroxy group, a (C₂-C₆)-hydroxyalkyl group, a (C₂-C₆)-polyhydroxyalkyl group, a (C₁-C₆)-alkoxy-(C₁-C₆)-alkyl group, an aryl-(C₁-C₆)-alkyl group, an amino group, a (C₁-C₆)-monoalkylamino group, a (C₁-C₆)-dialkylamino group, a dialkylaminoalkyl group —(CH₂)ₙ—NR′R″; wherein n is an integer from 2 to 6 and each of R′ and R″ is independently a linear or branched alkyl group which can optionally together form a ring.

4. The kit of claim 1 wherein the compound of formula II is selected from the group consisting of 2-(2-furoyl)acetonitrile, 2-(5-bromo-2-furoyl)acetonitrile, 2-(5-methyl-2-trifluoromethyl-3-furoyl)acetonitrile, 3-(2,5-dimethyl-3-furyl)-3-oxopropanitrile, 2-(2-thenoyl)acetonitrile, 2-(3-thenoyl)acetonitrile, 2-(5-fluoro-2-thenoyl)acetonitrile, 2-(5-chloro-2-thenoyl)acetonitrile, 2-(5-bromo-2-thenoyl)acetonitrile, 2-(5-methyl-2-thenoyl)acetonitrile, 2-(2,5-dimethylpyrrol-3-oyl)acetonitrile, 2-(1,2,5-trimethylpyrrol-3-oyl)acetonitrile, 1H-benzimidazol-2-ylacetonitrile, 1H-benzothiazol-2-ylacetonitrile, 2-(pyrid-2-yl)acetonitrile, 2,6-bis(cyanomethyl)pyridine, 2-(indol-3-oyl)acetonitrile, 2-(2-methylindol-3-oyl)acetonitrile, 8-cyanoacetyl-7-methoxy-4-methylcoumarin, 2-(2-isopropyl-5,6-benzoquinolin-4-oyl)acetonitrile, 2-(2-phenyl-5,6-benzoquinolin-4-oyl)acetonitrile, 2-(quinoxalin-2-yl)acetonitrile, 2-(coumaron-2-yl)acetonitrile, butyl 6,7-dichloro-5-(cyanoacetyl)-2,3-dihydro-1-benzofuran-2-carboxylate, 2-(6-hydroxy-4,7-dimethoxy-1-benzofuran-5-oyl)acetonitrile and 2-(1-phenyl-1,4-dihydrothiochromeno[4,3-c]pyrazol-3-oyl)acetonitrile.

5. The kit of claim 1 wherein R⁶ and R⁷ in formula (III) together with the nitrogen atom to which they are bonded form a saturated 5- or 6-membered ring.

6. The kit of claim 1 wherein the compound of the formulae (I), (II) and (III) is a salt selected from the group consisting of: 1,2-dihydro-1,3,4,6-tetramethyl-2-oxopyrimidinium; 1,2-dihydro-1,3,4-trimethyl-2-oxopyrimidinium; 1,2-dihydro-1,3,4,6-tetramethyl-2-thioxopyrimidinium; 1-allyl-1,2-dihydro-3,4,6-trimethyl-2-oxopyrimidinium; 1,2-dihydro-1-(2-hydroxyethyl)-3,4,6-trimethyl-2-oxopyrimidinium; 2-(Cyanomethyl)benzimidazole; 4,5-Dihydro-4-imino-2-(1-piperidinyl)thiazole; 4,5-Dihydro-4-imino-2-(4-morpholinyl)thiazole; 4,5-Dihydro-4-imino-2-(1-pyrrolidinyl)thiazole; wherein the counterion X⁻ of the salt is a physiologically compatible counterion.

7. The kit of claim 1 wherein the compound of the formula (IV) is selected from the group consisting of: 4-hydroxy-3-methoxybenzaldehyde, 3,5-dimethoxy-4-hydroxybenzaldehyde, 4-hydroxy-1-naphthaldehyde, 4-hydroxy-2-methoxybenzaldehyde, 3,4-dihydroxy-5-methoxybenzaldehyde, 3,4,5-trihydroxybenzaldehyde, 3,5-dibromo-4-hydroxybenzaldehyde, 4-hydroxy-3-nitrobenzaldehyde, 3-bromo-4-hydroxybenzaldehyde, 4-hydroxy-3-methylbenzaldehyde, 3,5-dimethyl-4-hydroxybenzaldehyde, 5-bromo-4-hydroxy-3-methoxybenzaldehyde, 4-diethylamino-2-hydroxybenzaldehyde, 4-dimethylamino-2-methoxybenzaldehyde, 2-methoxybenzaldehyde, 3-methoxybenzaldehyde, 4-methoxybenzaldehyde, 2-ethoxybenzaldehyde, 3-ethoxybenzaldehyde, 4-ethoxybenzaldehyde, 4-hydroxy-2,3-dimethoxybenzaldehyde, 4-hydroxy-2,5-dimethoxybenzaldehyde, 4-hydroxy-2,6-dimethoxybenzaldehyde, 4-hydroxy-2-methylbenzaldehyde, 4-hydroxy-2,3-dimethylbenzaldehyde, 4-hydroxy-2,5-dimethylbenzaldehyde, 4-hydroxy-2,6-dimethylbenzaldehyde, 3,5-diethoxy-4-hydroxybenzaldehyde, 2,6-diethoxy-4-hydroxybenzaldehyde, 3-hydroxy-4-methoxybenzaldehyde, 2-hydroxy-4-methoxybenzaldehyde, 2-ethoxy-4-hydroxybenzaldehyde, 3-ethoxy-4-hydroxybenzaldehyde, 4-ethoxy-2-hydroxybenzaldehyde, 4-ethoxy-3-hydroxybenzaldehyde, 2,3-dimethoxybenzaldehyde, 2,4-dimethoxybenzaldehyde, 2,5-dimethoxybenzaldehyde, 2,6-dimethoxybenzaldehyde, 3,4-dimethoxybenzaldehyde, 3,5-dimethoxybenzaldehyde, 2,3,4-trimethoxybenzaldehyde, 2,3,5-trimethoxybenzaldehyde, 2,3,6-trimethoxybenzaldehyde, 2,4,6-trimethoxybenzaldehyde, 2,4,5-trimethoxybenzaldehyde, 2,5,6-trimethoxybenzaldehyde, 2-hydroxybenzaldehyde, 3-hydroxybenzaldehyde, 4-hydroxybenzaldehyde, 2,3-dihydroxybenzaldehyde, 2,4-dihydroxy-benzaldehyde, 2,4-dihydroxy-3-methylbenzaldehyde, 2,4-dihydroxy-5-methylbenzaldehyde, 2,4-dihydroxy-6-methylbenzaldehyde, 2,4-dihydroxy-3-methoxybenzaldehyde, 2,4-dihydroxy-5-methoxybenzaldehyde, 2,4-dihydroxy-6-methoxybenzaldehyde, 2,5-dihydroxybenzaldehyde, 2,6-dihydroxybenzaldehyde, 3,4-dihydroxybenzaldehyde, 3,4-dihydroxy-2-methylbenzaldehyde, 3,4-dihydroxy-5-methylbenzaldehyde, 3,4-dihydroxy-6-methylbenzaldehyde, 3,4-dihydroxy-2-methoxybenzaldehyde, 3,5-dihydroxybenzaldehyde, 2,3,4-trihydroxybenzaldehyde, 2,3,5-trihydroxybenzaldehyde, 2,3,6-trihydroxybenzaldehyde, 2,4,6-trihydroxybenzaldehyde, 2,4,5-trihydroxybenzaldehyde, 2,5,6-trihydroxybenzaldehyde, 4-dimethylaminobenzaldehyde, 4-diethylaminobenzaldehyde, 4-dimethylamino-2-hydroxybenzaldehyde, 4-pyrrolidinobenzaldehyde, 4-morpholinobenzaldehyde, 2-morpholinobenzaldehyde, 4-piperidinobenzaldehyde, 3,5-dichloro-4-hydroxybenzaldehyde, 4-hydroxy-3,5-diiodobenzaldehyde, 3-chloro-4-hydroxybenzaldehyde, 5-chloro-3,4-dihydroxybenzaldehyde, 5-bromo-3,4-dihydroxybenzaldehyde, 3-chloro-4-hydroxy-5-methoxybenzaldehyde, 4-hydroxy-3-iodo-5-methoxybenzaldehyde, 2-methoxy-1-naphthaldehyde, 4-methoxy-1-naphthaldehyde, 2-hydroxy-1-naphthaldehyde, 2,4-dihydroxy-1-naphthaldehyde, 4-hydroxy-3-methoxy-1-naphthaldehyde, 2-hydroxy-4-methoxy-1-naphthaldehyde, 3-hydroxy-4-methoxy-1-naphthaldehyde, 2,4-dimethoxy-1-naphthaldehyde, 3,4-dimethoxy-1-naphthaldehyde, 4-dimethylamino-1-naphthaldehyde, 3-hydroxy-4-nitrobenzaldehyde, 2-hydroxy-3-methoxy-5-nitrobenzaldehyde, 5-nitrovanillin, 2,5-dinitrosalicylaldehyde, 5-bromo-3-nitrosalicylaldehyde, 2-dimethylaminobenzaldehyde, 2-chloro-4-dimethylaminobenzaldehyde, 4-dimethylamino-2-methylbenzaldehyde, 4-diethylaminocinnamaldehyde, 4-dibutylaminobenzaldehyde, 3-allyl-4-hydroxybenzaldehyde, 3-allyl-4-hydroxy-5-methoxybenzaldehyde, 3-allyl-4-hydroxy-5-methylbenzaldehyde, 3-allyl-5-bromo-4-hydroxybenzaldehyde, 3,5-diallyl-4-hydroxybenzaldehyde, 3-allyl-4-hydroxy-5-formylbenzaldehyde (5-allyl-4-hydroxy-isophthalaldehyde) and piperonal.

8. The kit of claim 1 wherein the pH is from 2 to 6.

9. The kit of claim 8 wherein the kit is free of coloring components.

10. The kit of claim 1 wherein the pH is from 8 to 11.

11. The kit of claim 10 wherein the kit is free of coloring components.

12. A method for the reversibly recoloring of keratin-containing fibers comprising the steps of (1) contacting fibers which have been colored previously with a colorant comprising, in a cosmetic carrier, component (A) comprising at least one CH-acidic compound selected from the group consisting of a compound of the formula I and/or enamine forms thereof, a compound of the formula (II), and a compound of the formula (III) 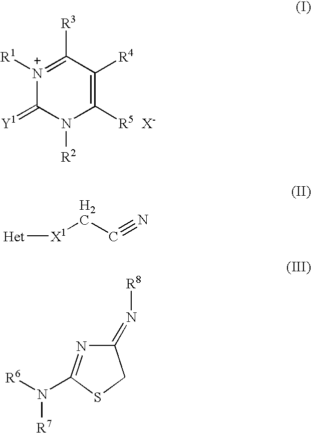
wherein
each of R¹ and R² is independently a linear or cyclic C₁-C₆-alkyl group, a C₂-C₆-alkenyl group, an optionally substituted aryl group, an optionally substituted heteroaryl group, an aryl-C₁-C₆-alkyl group, a C₁-C₆-hydroxyalkyl group, a C₂-C₆-polyhydroxyalkyl group, a C₁-C₆-alkoxy-C₁-C₆-alkyl group, a group R^{I}R^{II}N—(CH₂)ₚ—, in which each if R^{I} and R^{II} is independently a hydrogen atom, a C₁-C₄-alkyl group, a C₁-C₄-hydroxyalkyl group or an aryl-C₁-C₆-alkyl group, where R^{I} and R^{II}, together with the nitrogen atom, can form a 5-, 6- or 7-membered ring and p is a number 2, 3, 4, 5 or 6,
each of R³ and R⁵ is independently a hydrogen atom or a C₁-C₆-alkyl group, wherein at least one of the radicals R³ or R⁵ is a C₁-C₆-alkyl group,
R⁴ is a hydrogen atom, a C₁-C₆-alkyl group, a C₁-C₆-hydroxyalkyl group, a C₂-C₆-polyhydroxyalkyl group, a C₁-C₆-alkoxy group, a C₁-C₆-hydroxyalkoxy group, a group R^{III}RI^{V}N—(CH₂)_{q}—, wherein each of R^{III} and R^{IV} is independently a hydrogen atom, a C₁-C₆-alkyl group, a C₁-C₆-hydroxyalkyl group or an aryl-C₁-C₆-alkyl group and q is a number 1, 2, 3, 4, 5 or 6, where the radical R⁴, together with one of the radicals R³ or R⁵, can form a 5- or 6-membered aromatic ring which can optionally be substituted by a halogen atom, a C₁-C₆-alkyl group, a C₁-C₆-hydroxyalkyl group, a C₂-C₆-polyhydroxyalkyl group, a C₁-C₆-alkoxy group, a C₁-C₆-hydroxyalkoxy group, a nitro group, a hydroxy group, a group R^{V}R^{VI}N—(CH₂)₈—, wherein each of R^{V} and R^{VI} is independently a hydrogen atom, a C₁-C₆-alkyl group, a C₁-C₆-hydroxyalkyl group or an aryl-C₁-C₆-alkyl group and s is a number 0, 1, 2, 3, 4, 5 or 6,
Y¹ is an oxygen atom, a sulfur atom or a group NR^{VII}, wherein R^{VII} is a hydrogen atom, an aryl group, a heteroaryl group, a C₁-C₆-alkyl group or a C₁-C₆-arylalkyl group,
X⁻ is a physiologically compatible anion,
Het is an optionally substituted heteroaromatic,
X¹ is a direct bond or a carbonyl group,
each of R⁶ and R⁷ together with the nitrogen atom to which they are bonded form a saturated or unsaturated 5- or 6-membered ring or, independently of one another, are a (C₁-C₆)-alkyl group, a (C₂-C₆)-alkenyl group, an aryl group, an aryl-(C₁-C₆)-alkyl group, a (C₂-C₆)-hydroxyalkyl group, a (C₂-C₆)-polyhydroxyalkyl group or a group R^{I}R^{II}N—(CH₂)ₘ—, wherein each of R^{I} and R^{II} is independently a hydrogen atom, a (C₁-C₆)-alkyl group, a (C₁-C₆)-alkenyl group or an aryl-C₁-C₆-alkyl group, wherein R^{I} and R^{II}, together with the nitrogen atom to which they are bonded form a 5-, 6- or 7-membered ring and m is a number 2, 3, 4, 5 or 6, and
R⁸ is a hydrogen atom, a (C₁-C₆)-alkyl group, a (C₂-C₆)-alkenyl group, an aryl group, an aryl-(C₁-C₆)-alkyl group, a (C₂-C₆)-hydroxyalkyl group, a (C₂-C₆)-polyhydroxyalkyl group or a group R^{III}R^{IV}N—(CH₂)ₙ—, in which R^{III} and R^{IV}, independently of one another, are a hydrogen atom, a (C₁-C₆)-alkyl group, a (C₁-C₆)-alkenyl group or an aryl-C₁-C₆-alkyl group, where R^{III} and R^{IV}, together with the nitrogen atom, can form a 5-, 6- or 7-membered ring and n is a number 2, 3, 4, 5 or 6;
and component (B) comprising at least one aldehyde of the formula (IV) 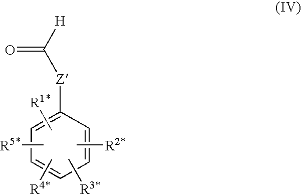
wherein
each of R^{1*}, R^{2*} and R^{3*} is independently a hydrogen atom, a halogen atom, a C₁-C₆-alkyl group, a hydroxy group, a C₁-C₆-alkoxy group, a C₁-C₆-dialkylamino group, a di(C₂-C₆-hydroxyalkyl)amino group, a di(C₁-C₆-alkoxy-C₁-C₆-alkyl)amino group, a C₁-C₆-hydroxyalkyloxy group, a sulfonyl group, a carboxy group, a sulfonic acid group, a sulfonamido group, a sulfonamide group, carbamoyl group, a C₂-C₆-acyl group or a nitro group,
Z′ is a direct bond or a vinylene group,
each of R^{4*} and R^{5*} is a hydrogen atom or together with the remainder of the molecule jointly form a 5- or 6-membered aromatic or aliphatic ring;
(2) rinsing the colored keratin-containing fibers from step (1) with a cosmetic composition having an acidic pH to form recolored fibers; (3) optionally, after a period of up to 4 weeks, recoloring the fibers from step (2) by rinsing with a cosmetic composition having an alkaline pH.

13. The method of claim 12 wherein when the cosmetic composition has an acidic pH the composition is not rinsed from the hair.

14. The method of claim 12 wherein when the cosmetic composition has an alkaline pH the composition is not rinsed from the hair.

15. The method of claim 12 wherein when the cosmetic composition has a pH of from 2 to 6.

16. The method of claim 15 wherein the cosmetic composition is free of coloring components.

17. The method of claim 12 wherein when the cosmetic composition has a pH of from 8 to 11.

18. The method of claim 17 wherein the cosmetic composition is free of coloring components.
